(19) United States
(12) Patent Application Publication
(10) Pub. No.: US 2023/0096810 A1
(43) Pub. Date: Mar. 30, 2023

(54) DOSAGE FORMS FOR THE DELIVERY OF A PROBIOTIC

(71) Applicant: Johnson & Johnson Consumer Inc., Skillman, NJ (US)

(72) Inventors: Anthony R. Geonnotti, III, Princeton, NJ (US); Patricia L. Golas, Somerville, NJ (US); Richard Besingi, Pennington, NJ (US); Shoba Pillai, Newtown, PA (US)

(21) Appl. No.: 17/933,174

(22) Filed: Sep. 19, 2022

Related U.S. Application Data
(60) Provisional application No. 63/261,581, filed on Sep. 24, 2021.

(51) Int. Cl. A23L 29/269; A23L 33/15; A23L 33/00; A23L 33/16

(52) U.S. Cl. CPC A23L 29/269; A23L 33/15; A23L 33/40; A23L 33/16

(57) ABSTRACT

Provided are compositions and methods for oral delivery to an infant of a composition containing Bifidobacterium. The composition may be pre-loaded into or placed directly onto an artificial nipple or pacifier and may also be applied directly to the skin of a caregiver and/or to one of the gums, teeth or cheek of the infant. The compositions of the invention are provided in soft gel fill, dissolvable or disintegrable tablet or powder or semisolid forms and are stable for at least 24 months when stored at 25° C. and 65% relative humidity.

This application claims the benefit of U.S. application 63/261,581 filed on Sep. 24, 2021, the complete disclosure of which is hereby incorporated herein by reference for all purposes.

### FIELD

The present invention generally relates to dosage forms and methods for the delivery of an effective amount of a probiotic including Bifidobacterium to a patient.

### BACKGROUND

Probiotics are live microorganisms that are intended to have health benefits when consumed or applied to the body. Probiotics may be administered to address a myriad of health conditions including infantile digestive health, colic, and sleep. Probiotics are also increasingly administered to support immune health and prevent atopic diseases, including atopic dermatitis and food allergy (Sestito et al. Front. in Pediatr. 22 Dec. 2020; Henrick et al. Cell 22 Jul. 2021 184(15): p. 1-15).

Probiotics are found in infant formulas, supplements and food products for babies or children. These probiotics typically consist of Bifidobacterium, including the species B. longum, B. breve, B. bifidum, B. pseudocatenulatum, B. globosum, B. adolescentis, B. moukalabense, B. reuteri, B. pseudolongum, B. dentium, B. catenulatum, B. sp002742445, B. callitrichos, B. scardovii, B. tissieri, B. subtile, B. gallinarum, B. choerinum, B. angulatum, B. primatium, B. myosotis, B. mongoliense, B. merycicum, B. lemurum, B. stellenboschense, B. scaligerum, B. saguini, B. pullorum, B. felsineum, B. eulemuris, B. cuniculi, B. callitrichos_A, B. biavatii, B. anseris, B. vansinderenii, B. sp900551485, B. sp003952945, B. sp003952025, B. sp003952005, B. simiarum, B. pseudolongum_C, B. parmae, B. margollesii, B. kashiwanohense_A, B. italicum, B. imperatoris, B. cricetid, B. catulorum, B. callitrichidarum, B. animalis, B. aesculapii, and combinations thereof. This is because certain Bifidobacterium have the ability to break down complex oligosaccharides that are found in human milk (see U.S. Pat. No. 8,198,872 and US Pub. No. 2013/01958031; Sela et al, 2008, PNAS, 105(48): p. 18964-69). The suitable Bifidobacterium may be those having at least one human milk oligosaccharides (HMO) gene cluster.

The fermentation product from Bifidobacterium production may be concentrated and freeze dried to provide a concentrated powder. The powder may be packaged in a sachet or suspended in an edible oil. U.S. Pat. No. 10,716,8176 describes a method for obtaining an active Bifidobacterium composition.

One example of a product containing a probiotic is Similac® Probiotic Tri-Blend available from Abbott (Abbott Park, Ill.) which is a powder containing Bifidobacterium lactis, Bifidobacterium infantis and Streptococcus thermophilus. Another example is Evivo® available from Evolve BioSystems (Davis, Calif.) which contains Bifidobacterium infantis. Evivo is available as a powder or a medium-chain triglyceride oil (MCT oil) suspension.

Bifidobacterium and other probiotics decrease in potency over time and this decrease can be caused by exposure to certain temperatures, moisture and oxygen. This leads to a short “shelf-life” or expiration date for products and difficulties and expense in formulation, transportation and storage. Many current probiotics are shipped using a cold chain transport system because the probiotics must be maintained at or below 25 degrees Celsius (77 degrees Fahrenheit) from production to delivery to a patient. Accordingly, there is an ongoing need for probiotic dosage forms that are resistant to moisture and oxygen and can be transported and stored more easily.

Bifidobacterium and other probiotics may be most effective for their intended purpose when co-administered with a prebiotic, vitamins or supplements and/or food allergen or food introduction products. Accordingly, there is an ongoing need for stable dosage forms that include both a probiotic and one or more additional components and/or kits that include both a probiotic and one or more additional components.

### SUMMARY OF THE INVENTION

The Bifidobacterium of the present invention and/or combinations of Bifidobacterium with other probiotics may be incorporated into several different types of dosage forms. These forms include soft gelatin capsules or softgels, wherein the Bifidobacterium is incorporated into the fill material prior to encapsulation in a gelatin based shell or capsule. The shell may also be comprised of a shell material which dissolves in liquid other than gelatin, such as a polymer or those derived from starch. Suitable polymers for a shell may include hypromellose, hydroxypropyl cellulose, polymethacrylate(s), pullulan and methylcelllose. The soft gel may include a twist off portion, cap or feature to facilitate opening, so as to expose the fill for direct ingestion, incorporation into a food or other edible medium, or for application to the skin of a caregiver, to the oral cavity, cheek or gums of an infant or to the surface of a delivery device such as an artificial nipple or pacifier.

The Bifidobacterium may also be incorporated into an edible semisolid for ingestion or direct application to the skin. This edible semisolid may be in the form of an emulsion, topical gel, cream, paste, ointment or balm and incorporate at least one lipid material. This edible semisolid may be fully anhydrous. In specific applications the semisolid is applied to the skin for the ingestion of the Bifidobacterium during infant nursing. This application method may comprise different steps. The composition may be applied directly to the nipple or surrounding skin area of the caregiver or parent. The composition may also be mixed or diluted in a liquid such as infant formula or breastmilk and subsequently applied to the nipple or surrounding skin area. The composition may also be applied or pre-applied, e.g. loaded into a pacifier or artificial nipple for ingestion. In another application, the pacifier or artificial nipple may comprise one portion of the composition such as a prebiotic, wherein the probiotic is administered in a separate portion. This separate portion could be provided through direct oral administration to an infant, or through application to the nipple, surrounding skin or finger. In other specific applications, the topical gel can be placed on a caregiver's finger and applied directly to the infant's gums or cheek.

The Bifidobacterium may also be incorporated into a solid powder or tablet that is substantially free of lactose. As defined herein free of lactose or lactose-free is defined as less than 1.0 percent, e.g. less than 0.2 percent of lactose by weight of the form. Solid dosage forms of the present invention may also include dispersible tablets, or tablets that can be placed into a liquid medium and dissolved prior to administration. Solid dosage forms of the present invention may also include dissolvable tablets which can be dissolved in a liquid or semisolid medium prior to administration. Other solid dosage forms include powder compositions which can be incorporated directly into a liquid or semisolid medium prior to administration. These powder compositions may be packaged as individual dose(s) in a sachet, pouch, bottle or blister prior to administration. The powder composition may also be incorporated in a hard shell capsule or within a dissolvable capsule shell, which can be opened manually or dissolved in a liquid or semisolid prior to administration. Semisolids for the purpose of incorporating the composition or the present invention may include creams, oils, or pureed or blended foods comprising fruits and vegetables.

For solid dosage forms that are free of lactose, an alternative inert powder carrier may be utilized. Suitable inert powder carriers include starch(es), cellulose(s), sugars and sugar alcohols.

In some examples, the Bifidobacterium is incorporated into dosage forms that contain an antioxidant such as vitamin C, vitamin E, beta-carotene, cysteine, propyl gallate, butylated hydroxytoluene (BHT).

In some examples, the Bifidobacterium containing dosage forms include a prebiotic such as an oligosaccharide or synthetic equivalent.

In some examples, the Bifidobacterium containing dosage forms include a vitamin, supplement or mineral.

In some examples, the Bifidobacterium containing dosage forms include a food allergen or food introduction product such as an allergen selected from the group of cow milk, egg, peanut, wheat, soy, sesame, fish, shellfish and tree nut.

These and other features and advantages of the present invention will be readily apparent from the following detailed description of the invention.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

As used herein, the term “probiotic” refers to live microorganisms which when administered in adequate amounts confer a health benefit on the host. These probiotic strains generally have the ability to survive the passage through the upper part of the digestive tract. They are non-pathogenic, non-toxic and exercise their beneficial effect on health on the one hand via ecological interactions with the resident flora in the digestive tract, and on the other hand via their ability to influence the immune system positively. Depending on the composition of probiotics, these microorganisms, when given in a sufficient number, have the ability to progress live through the intestine, however they do not cross the intestinal barrier and their primary effects are therefore induced in the lumen and/or the wall of the gastrointestinal tract. They then form part of the resident flora during the administration period. This colonization (or transient colonization) allows the probiotic microorganisms to exercise a beneficial effect, such as the repression of potentially pathogenic micro-organisms present in the flora and interactions with the immune system of the intestine.

As used herein, the term “effective amount” means an amount sufficient to induce the desired effect. The term “safe amount” means an amount that is low enough to avoid serious side effects. The safe and/or effective amount of the compound, extract, or composition will vary with, e.g., the age, health and environmental exposure of the end user, the duration and nature of the treatment, the specific extract, ingredient, or composition employed, the particular pharmaceutically-acceptable carrier utilized, and like factors.

As used herein, “essentially free” or “substantially free” of an ingredient means containing less than 0.1 weight percent, or less than 0.01 weight percent, or none of an ingredient. Unless otherwise indicated, percentages used to express amounts of ingredients are percentage by weight (referred to as “weight %”, “wt %”, “% by weight” or “% (w/volume)”). Similarly, weight ratios used to express relative proportions of ingredients are also determined using percentage by weight (i.e., weight ratios are calculated by dividing the percentage by weight of one ingredient by another).

Composition

The Bifidobacterium species may include B. longum, B. breve, B. bifidum, B. pseudocatenulatum, B. globosum, B. adolescentis, B. moukalabense, B. reuteri, B. pseudolongum, B. dentium, B. catenulatum, B. sp002742445, B. callitrichos, B. scardovii, B. tissieri, B. subtile, B. gallinarum, B. choerinum, B. angulatum, B. primatium, B. myosotis, B. mongoliense, B. merycicum, B. lemurum, B. stellenboschense, B. scaligerum, B. saguini, B. pullorum, B. felsineum, B. eulemuris, B. cuniculi, B. callitrichos_A, B. biavatii, B. anseris, B. vansinderenii, B. sp900551485, B. sp003952945, B. sp003952025, B. sp003952005, B. simiarum, B. pseudolongum_C, B. parmae, B. margollesii, B. kashiwanohense_A, B. italicum, B. imperatoris, B. cricetid, B. catulorum, B. callitrichidarum, B. animalis, B. aesculapii, and combinations thereof.

The Bifidobacterium may be formulated into a composition which is easy to use and allows for consistent dosing. The fermentation product from Bifidobacterium production may be concentrated and freeze dried to provide a concentrated powder. The composition may contain about 1 million, 500 million, 1 billion, 2 billion, 3 billion, 4 billion, 5 billion, 6 billion, 7 billion, 8 billion, 9 billion, 10 billion or 12 billion to about 8 billion, 9 billion, 10 billion, 20 billion, 30 billion, 40 billion, 50 billion, 60 billion, 70 billion, 80 billion, 90 billion, 100 billion, 200 billion, 250 billion or 500 billion colony forming units (CFU) of Bifidobacterium per gram dry weight.

The Bifidobacterium may also be formulated with a prebiotic. As used herein, the term “prebiotic” refers to a substance that is indigestible to a host, but can be metabolized by a microorganism, and once metabolized can promote the growth of that microorganism within a host. Prebiotic refers to an ingredient that allows specific changes in the composition and/or activity in the gastrointestinal microbiota. In some examples, a prebiotic can be a comestible food or beverage or ingredient thereof. Prebiotics can include complex carbohydrates, amino acids, peptides, minerals, or other essential nutritional components for the survival of the bacterial composition. Prebiotics include, but are not limited to, amino acids, biotin, fructooligosaccharide, galactooligosaccharides, hemicelluloses (e.g., arabinoxylan, xylan, xyloglucan, and glucomannan), inulin, chitin, lactulose, mannan oligosaccharides, oligofructose-enriched inulin, gums (e.g., guar gum, gum arabic and carregenaan), oligofructose, oligodextrose, tagatose, resistant maltodextrins (e.g., resistant starch), trans-galactooligosaccharide, pectins (e.g., xylogalactouronan, citrus pectin, apple pectin, and rhamnogalacturonan-I), dietary fibers (e.g., soy fiber, sugarbeet fiber, pea fiber, corn bran, and oat fiber), xylooligosaccharides and any other glycans or compounds consisting of multiple monosaccharides linked glycosidically.

Prebiotics that can be metabolized by Bifidobacterium include oligosaccharides. As used herein, the term “oligosaccharide” refers to a saccharide polymer containing 2 to 20, 2 to 10, 3 to 20 or 3 to 10 monosaccharide units. The oligosaccharide may be those naturally found in a mammalian milk (e.g., human, or bovine) known as human milk oligosaccharides, human milk glycans or “HMOs.” The oligosaccharide may be synthesized. There are a number of commercially available HMOs including Glycan™ products offered by DSM Nutritional Products AG. Prebiotics that can be metabolized by Bifidobacterium also include synthetic molecules that are structurally distinct from yet functionally equivalent to natural HMOs such as lacto-N-tetraose (LNT) and 2′-fucosyllactose (2′-FL).

The composition containing the Bifidobacterium may also contain an auxiliary component. Such auxiliary component are those commonly used in the art and may be selected from metabolites, flow agents or combinations thereof. Examples of flow agents include starch, silicon dioxide, cellulose, sodium bicarbonate, calcium silicate and the like. The auxiliary component may also be a milk protein or constituent. The auxiliary component may comprise lactose. That is, in such an example, the Bifidobacterium is in powder form mixed with lactose and derivatives thereof. Lactose derivatives may include lactulose, lactitol, lactobionic acid, galacto-oligosaccharides (GOS), lactose monohydrate and tagatose.

The composition may also be substantially free of lactose to mitigate lactose sensitivity. Suitable lactose substitutes include starch(es), cellulose(s), sugars and sugar alcohols.

The composition containing the Bifidobacterium may also contain an antioxidant. Examples of antioxidants include vitamin C, vitamin E, beta-carotene, cysteine, propyl gallate, butylated hydroxytoluene (BUT). The final form of the composition containing the antioxidant can be any known in the art. As described above, the Bifidobacterium may be in dried form (e.g., spray-dried or freeze-dried) as a powder. Said powder may be dosed as a packet, sachet, tablet, foodstuff, capsule, lozenge, tablet, suspension, dry form, etc.

The final form of the composition may additionally comprise a vitamin, supplement or mineral. Suitable vitamins, supplements and minerals may include but are not limited to vitamin B, vitamin C, vitamin D, vitamin E, vitamin K, iron, omega 3 fatty acids such as eicosapentaenoic acid (EPA) and docosahexaenoic acid (DHA) essential fatty acids. magnesium salts, calcium salts and potassium salts.

The final form of the composition may additionally comprise a food allergen or food introduction product. Suitable food allergens include cow milk, egg, peanut, wheat, soy, sesame, fish, shellfish and tree nut.

The final form of the composition may be stable up to at least 24 months when stored at 25° C. and 65% relative humidity (RH), or at least 12 months when stored at 25° C. and 65% relative humidity (RH), or at least 6 months when stored at 25° C. and 65% relative humidity (RH) or at least 3 months when stored at 40° C. and 75% relative humidity (RH), or at least 1 month when stored at 40° C. and 75% relative humidity (RH), or at least 6 months when stored at 50° C.

The final form of the composition may not comprise added sugar, or may be substantially free of added sugar, or be defined as comprising “no added sugars”. If a prebiotic is added to the composition and wherein the prebiotic portion comprises a carbohydrate or sugar, the probiotic portion may be separate and may not comprise added sugars or be substantially free of added sugar.

The excipients of the final form of the composition may be derived directly from plants or sourced directly from plants, so as to be defined as “plant-based”. The excipients of the final form of the composition may also be sourced from non-genetically modified organisms (“non-GMO”). At least 95% of the excipients of the final form of the composition may be plant-based or non-GMO.

### Emulsion or Edible Semi-Solid

The emulsion or semi-solid composition containing the Bifidobacterium may also contain ingredients suitable for skin in need of improving skin barrier function and moisturization or for soothing or protecting against skin irritation such as hyaluronic acid, glycerin, petrolatum and propylene glycol. As used herein, “skin in need of improving skin barrier function and moisturization” means skin that is, but not limited to, lacking in moisture, lacking in sebum, cracked, dry, itchy, scaly, xerodermic, dehydrated, lacks suppleness, lacks radiance, dull, or lacks lipids.

Various oils may be used as a carrier within the emulsion or edible semi-solid composition comprising the Bifidobacterium. Carriers may include but are not limited to vegetable oils, fish oil, olive oil, soy oil, nut oil, mineral oil, and avocado oil. Additional carriers include glyceryl behenate and glyceryl stearate. In composition of an edible oil, the composition may comprise 5 percent to 95 percent by weight of the carrier, e.g. 30 percent to about 90 percent by weight of the carrier.

As used herein, the term “emulsifier” refers to an ingredient that helps keep ingredients (such as oil and water) from separating in an emulsion. The composition may comprise emulsifiers such as waxes, beeswax, microcrystalline wax, lecithin, esters (PGE), polysorbates, stearoyl lactylates, propylene glycol esters (PGMS), and sucrose esters.

The emulsion or edible semi-solid of the present invention may be added to a soft gel. The soft gel may be for direct administration, reconstitution in a liquid, or as a carrier for administration to a skin surface. In some examples, the soft gel form has a twist-off portion, cap or feature so as to expose the emulsion or edible semi-solid fill for direct ingestion, incorporation into a food or other edible medium, or for application to the skin of a caregiver, to the oral cavity, cheek or gums of an infant or to the surface of a delivery device such as an artificial nipple or pacifier.

The portion of the composition which comprises the Bifidobacterium infantis may be provided in the form of a nanoemulsion or coated particle, so as to protect the Bifidobacterium infantis from oxygen, water or the surrounding composition. Suitable coating technologies include microencapsulation compositions, such as those documented in U.S. Pat. No. 11,433,107 documented herein by reference. Suitable nanoemulsion compositions include but are not limited to those types documented in U.S. Pat. No. 8,993,019 documented herein by reference.

### Solid Form

The Bifidobacterium may be added to the composition and delivered as a solid form. As used herein, “solid form” may be defined as an orally disintegrating tablet, a dissolvable tablet or powder composition. The solid form may be administered directly to an infant, or dissolved, or mixed in another composition such as a liquid or semi-solid prior to administration.

The solid form may comprise various excipients. Examples of suitable excipients include, but are not limited to, fillers, adsorbents, disintegrants, lubricants, glidants, sweeteners, superdisintegrants, flavor and aroma agents, antioxidants, preservatives, texture enhancers, and mixtures thereof. One or more of the above ingredients may be present on the same particle of the powder blend.

Suitable fillers include, but are not limited to, carbohydrates (as discussed herein) and water insoluble plastically deforming materials (e.g., microcrystalline cellulose or other cellulosic derivatives), and mixtures thereof.

Suitable disintegrants include, but are not limited to, sodium starch glycolate, cross-linked polyvinylpyrrolidone, cross-linked carboxymethylcellulose, starches, microcrystalline cellulose, and mixtures thereof.

Suitable lubricants include, but are not limited to, long chain fatty acids and their salts, such as magnesium stearate and stearic acid, talc, glycerides waxes, and mixtures thereof.

Suitable glidants include, but are not limited to, colloidal silicon dioxide.

Examples of sweeteners include, but are not limited to, synthetic or natural sugars; artificial sweeteners such as saccharin, sodium saccharin, aspartame, acesulfame, thaumatin, glycyrrhizin, sucralose, dihydrochalcone, alitame, miraculin, monellin, and stevside; sugar alcohols such as sorbitol, mannitol, glycerol, lactitol, maltitol, and xylitol; sugars extracted from sugar cane and sugar beet (sucrose), dextrose (also called glucose), fructose (also called laevulose), and lactose (also called milk sugar); isomalt, salts thereof, and mixtures thereof.

Examples of superdisintegrants include, but are not limited to, croscarmellose sodium, sodium starch glycolate and cross-linked povidone (crospovidone). In one embodiment the tablet contains up to about 5% by weight of such superdisintegrant.

Examples of flavors and aromatics include, but are not limited to, essential oils including distillations, solvent extractions, or cold expressions of chopped flowers, leaves, peel or pulped whole fruit containing mixtures of alcohols, esters, aldehydes and lactones; essences including either diluted solutions of essential oils, or mixtures of synthetic chemicals blended to match the natural flavor of the fruit (e.g., strawberry, raspberry and black currant); artificial and natural flavors of brews and liquors, e.g., cognac, whisky, rum, gin, sherry, port, and wine; tobacco, coffee, tea, cocoa, and mint; fruit juices including expelled juice from washed, scrubbed fruits such as lemon, orange, and lime; spear mint, pepper mint, wintergreen, cinnamon, cacoe/cocoa, vanilla, liquorice, menthol, eucalyptus, aniseeds nuts (e.g., peanuts, coconuts, hazelnuts, chestnuts, walnuts, colanuts), almonds, raisins; and powder, flour, or vegetable material parts including tobacco plant parts, e.g., genus Nicotiana, in amounts not contributing significantly to the level of nicotine, and ginger.

Examples of antioxidants include, but are not limited to, tocopherols, ascorbic acid, sodium pyrosulfite, butylhydroxytoluene, butylated hydroxyanisole, edetic acid, and edetate salts, and mixtures thereof.

Examples of preservatives include, but are not limited to, citric acid, tartaric acid, lactic acid, malic acid, acetic acid, benzoic acid, and sorbic acid, and mixtures thereof.

In one embodiment, the solid form contains at least one carbohydrate. The carbohydrate can contribute to the dissolvability and mouth feel of the tablet, aid in distributing the meltable binder across a broader surface area, and diluting and cushioning the pharmaceutically active agent. Examples of carbohydrates include, but are not limited to, water-soluble compressible carbohydrates such as sugars (e.g., dextrose, sucrose, maltose, isomalt, and lactose), starches (e.g., corn starch), sugar-alcohols (e.g., mannitol, sorbitol, maltitol, erythritol, lactitol, and xylitol), and starch hydrolysates (e.g., dextrins, and maltodextrins).

### Packaging

The final form of the composition may be packaged into a blister, bottle, vial or sachet and may be packaged under nitrogen. Alternatively the composition may be packaged into a dispenser which does not allow for the composition to come in contact with oxygen or air, or allow for the introduction of air upon dispensing. This type of dispensing may be achieved with dispensers such as those supplied by Aptar corporation (Aptarinc), marketed under their “Airless” pump technologies, also documented in U.S. Pat. No. 11,213,843, and documented herein by reference.

### Prophetic Example—Soft Gel

TABLE 1
Soft Gel Fill Formulation

| | Batch | |
|---|---|---|
| Ingredient | Weight (g) | Mg/Dose |
| Bifidobacterium infantis (monocoated with | | 8 B CFUs* |
| polydiglyceryl distearate) | | |
| Soy Oil | 893.0 | 350.0 |
| Soy Lechitin | 5.1 | 2.0 |
| Beeswax | 102.0 | 40.0 |
| TOTAL | 1000.0 | 392.0 |

*Equivalent to 8 Billion CFU counts per dose; not included in batch weight

1. The Bifidobaacterium infantis are monocoated with polyglyceryl distearate (Plurol Stearique WL1009) according to the process described in Italian Patent Application No. RM2009A000104
2. For the suspension, beeswax is melted in soy oil at about 65° C. Soy lecithin is then added to the mixture and the combined formulation cooled at less than or about 25° C.
3. The microencapsulated probiotic bacteria were suspended in the cooled suspending formulation of soy oil, bees wax and soy lecithin and mixed at less than 25° C. for 10 minutes.
4. The mixed fill is milled while maintaining the temperature below 25° C. for 10 minutes, while under vacuum and under nitrogen to prevent oxidation of the formulation.
5. The milled fill is then encapsulated in a soft gelatin capsule using a standard rotary die encapsulation machine as follows: the milled fill and the shell material were loaded into separate receivers connected to the machine. The machine prepared from the molten shell material two bands of solid ribbons, which are cooled and lubricated with a mixture of medium chain triglycerides and lecithin. The bands are directed into the position of two rotating dies having specific pockets of the required size and shape for formation of the capsule. The continuing contra-rotation of the opposed dies form a seal between the two ribbons contacting the dies while the fill material is simultaneously injected into the body of the capsule so formed. The continuing rotation of the dies cuts the newly formed capsule from the ribbon.

### Prophetic Example—Semisolid

TABLE 2
Edible Semisolid Formulation

| | Batch | |
|---|---|---|
| Ingredient | Weight (g) | Mg/Dose |
| Bifidobacterium infantis* | | 8 B CFUs* |
| Coconut Oil | 450.0 | 225.0 |
| Shea Butter | 450.0 | 225.0 |
| Corn Starch | 100.0 | 50.0 |
| TOTAL | 1000.0 | 500.0 |

*Equivalent to 8 Billion CFU counts per dose; not included in batch weight

Blending: The blend(s) is prepared as follows (according to formula in Table 2):
1. The coconut oil, shea butter are added to a suitable vessel and preblended until sufficiently uniform at 60° C. The corn starch is added while mixing at 100 RPM.
2. The mixture is cooled to 25° C.
3. The Bifidobacterium infantis is added to the mixture from Step 2 and mixed at 25° C. until uniform at relatively low speed (less than 100 RPM).
4. The blend is added to individual vials under nitrogen and capped.

### Prophetic Example(s)—Dissolvable/Disintegratable Compressed Tablets

TABLE 3
Dissolvable/Disintegratable Tablet Formulation A

| | | Batch | | |
|---|---|---|---|---|
| | Commercial | Weight | | % |
| Ingredient | Supplier | (g) | Mg/Tag | w/w |
| Bifidobacterium infantis | | | 8 B CFUs* | |
| Mannitol USP & Starch | Roquette | 745.0 | 447.0 | 74.5 |
| NF (Pearlitol Flash) | | | | |
| Lactose Monohydrate NF | Roquette | 200.0 | 120.0 | 20.0 |
| Silicified | FMC | 50.0 | 30.0 | 5.0 |
| Microcrystalline | | | | |
| Cellulose | | | | |
| Magnesium Stearate | Mallincrodt | 5.0 | 3.0 | 0.5 |
| | Inc. | | | |
| Total | | 1000.0 | 600.0 | 100.0 |

*Equivalent to 8 Billion CFU counts per dose; not included in batch weight

### Dissolvable/Disintegratable Tablet Formula A

Part A: Blending: The blend(s) is prepared as follows (according to formula in Table 3):
1. The lactose monohydrate and a portion of the Pearlitol Flash are preblended until sufficiently uniform in a V-Blender. Pearlitol Flash is commercially available from the Roquette Corporation.
2. The other materials including the Bifidobacterium infantis, except the magnesium stearate and a small portion of the Pearlitol Flash, are added and blended for an additional period.
3. The magnesium stearate and remaining Pearlitol Flash are passed through a 30 mesh screen and added and blended for additional 5 minutes.

Part B: Compression: The blend is compressed into tablets using round tooling to a hardness of 5.0 kP

TABLE 4
Dissolvable/Disintegratable Tablet Formulation B (Lactose Free)

| | | Batch | | |
|---|---|---|---|---|
| | Commercial | Weight | | % |
| Ingredient | Supplier | (g) | Mg/Tag | w/w |
| Bifidobacterium infantis | | 4.0 | 8 B CFUs* | 0.8 |
| | | | (12.8 mg) | |
| Mannitol (Sweetpearl | Roquette | 454.7 | 1454.9 | 90.9 |
| P300 DC) | | | | |
| Sucralose | Tate & Lyle | 0.75 | 2.4 | 0.15 |
| Acesulfame K | Nutrinova | 0.25 | 0.8 | 0.05 |
| Hydroxypropylcellulose | Ashland | 12.50 | 40.0 | 2.50 |
| (Klucel Nutra D) | Specialty | | | |
| Flavor | | 20.3 | 65.1 | 4.1 |
| Magnesium Stearate | Mallincrodt | 7.5 | 24.0 | 1.5 |
| | Inc. | | | |
| | | 500.0 g | 1600 mg | 100.0 |

*Colony Forming Units

### Dissolvable/Disintegratable Tablet Formula B (Lactose Free)

Part A: Blending: The blend(s) is prepared as follows (according to formula in Table 4):
1. The mannitol, sucralose, Acesulfame K. hydroxypropylcellulose, flavor and Bifidobacterium infantis are passed through a 60 mesh screen to de-lump the materials.
2. The materials from step 1 are placed into a suitable V-blender and blended for 10 minutes.
3. The magnesium stearate is screened through a 30 mesh screen and added and blended for additional 5 minutes. The final blend is discharged into a plastic bag.

Part B: Compression: The blend is compressed into tablets using round tooling to a hardness of 5.0 kP.

### Administration of Bifidobacterium or Combination of Bifidobacterium and Other Components

As used herein, the term “administering” refers to providing a given dose of Bifidobacterium to infants as part of their feeding routine (i.e., it is used as a food supplement). The Bifidobacterium may be administered to the infant in the dosage form provided or mixed with any medium that can be consumed by the infant, including breast milk, infant formula, water or food prior to administering the Bifidobacterium to the infant.

In the case of mixing with a medium prior to administration, the Bifidobacterium may be mixed into breastmilk. Alternatively, the Bifidobacterium may be mixed into infant formula prior to administering the Bifidobacterium to the breastfed infant. The Bifidobacterium is mixed with enough infant formula or breastmilk so that the infant is able to completely incorporate the Bifidobacterium and so that the infant is still likely and able to consume the entire dose of Bifidobacterium. Thus, the Bifidobacterium may be mixed with about 3 to about 5 mL of breastmilk or infant formula prior to administering the Bifidobacterium to the breastfed infant. The Bifidobacterium composition may be mixed by any suitable means, including simply stirring (or any other suitable means to obtain a mixture) the composition with the medium (e.g., infant formula, breast milk, water) in a bowl. The composition mixed with infant formula or breastmilk may then be fed to the infant by any suitable means. Suitable means of feeding to the infant include use of a feeding syringe, spoon, or bottle. The Bifidobacterium may be administered prior to feeding the infant when the infant is more likely to be hungry, which is thought to increase the likelihood of the infant consuming the entirety of the dose.

The Bifidobacterium dosage form may be administered to the infant directly without mixing with breast milk, formula or water. The Bifidobacterium dosage form may be applied directly to the mother's skin prior to breastfeeding. The Bifidobacterium dosage form may be applied directly to a bottle nipple or pacifier prior to feeding. The Bifidobacterium dosage form may be applied to a finger or flexible dosing device and applied directly inside the infant's mouth on the gums or cheeks.

The composition may also be applied or pre-applied, e.g. loaded into a pacifier or artificial nipple for ingestion. In another application, the pacifier or artificial nipple may comprise one portion of the composition such as a prebiotic, wherein the probiotic is administered in a separate portion. This separate portion could be provided through direct oral administration to an infant, or through application to the nipple, surrounding skin or finger.

The dose and dosing frequency may be selected as desired. For example, the Bifidobacterium may be administered once daily. In such an example, the dose once daily may contain from about 5-15 billion or about 8 billion CFU. Splitting the total desired dose into smaller doses is also contemplated. Examples could include smaller doses several times throughout the day (e.g., 2, 3, 4 or 5 times per day).

The total dose given per day may range from about 1 million, 500 million, 1 billion, 2 billion, 3 billion, 4 billion, 5 billion, 6 billion, 7 billion, 8 billion, 9 billion, 10 billion or 12 billion to about 8 billion, 9 billion, 10 billion, 20 billion, 30 billion, 40 billion, 50 billion, 60 billion, 70 billion, 80 billion, 90 billion, 100 billion, 200 billion, 250 billion or 500 billion colony forming units (CFU) of the Bifidobacterium. The total dose given per day may range from about 5 to about 15 billion CFU, or be about 8 billion CFU. Such total dose values may be given in one dose.

The Bifidobacterium may be administered beginning on the 1^{st}, 2^{nd}, 3^{rd}, 4^{th}, 5^{th}, 6^{th} day or first week of life, or beginning within the first 2, 3, 4, 5, 6, 7, 8, 9, 10, 11 or 12 weeks of life, or beginning with the first 3, 4, 5, 6, 7, 8, 9, 10, 11 or 12 months of life. As used herein the term “of life” means after birth. Once started, the Bifidobacterium may continue to be administered until the 4th, 5th, 6th, 7th, 8th, 9th, 10th, 11th, 12th week of life, or until the 3rd, 4th, 5th, 6th, 7th, 8th, 9th, 10th, 11th or 12th month of life. The Bifidobacterium may be first administered within the first 2 weeks of life. The Bifidobacterium may be first administered within the first 2 weeks of life and until the 12th week of life.

The infants may be breastfed infants or formula fed infants or combinations of both. As used herein, the term “breastfed” means that the infant derives at least some of its sustenance from human breastmilk. The infant may either nurse or the breastmilk may be expressed (e.g., pumped or hand-expressed) and given to the infant. The breastfed infant may be at least about 50, 60, 75, 80, 90% or 95% breastfed. The remainder of the infant's sustenance may be derived from infant formula or other food. Alternatively, the breastfed infant may be exclusively breastfed. As used herein, the term “exclusively breastfed” means that the infant does not receive infant formula, except that small amounts of infant formula may be used for the sole purpose to mix with the Bifidobacterium and administer to the infant. For breastfed infants, any caloric contribution from other sources during the first 3 months of life, including medicines, the Bifidobacterium composition, or any medium used to deliver the Bifidobacterium, etc. may be considered negligible. In the case of formula fed infants, the Bifidobacterium dosage form contains prebiotics such as oligosaccharides.

While the foregoing description represent exemplary embodiments of the present invention, it will be understood that various additions, modifications, and substitutions may be made herein without departing from the spirit and scope of the present invention. In particular, it will be clear to those skilled in the art that the present invention may be embodied in other specific forms, structures, arrangements, proportions, and with other elements, materials, and components, without departing from the spirit or essential characteristics thereof. One skilled in the art will appreciate that the invention may be used with many modifications of structure, arrangement, proportions, materials, and components and otherwise, used in the practice of the invention, which are particularly adapted to specific environments and operative requirements without departing from the principles of the present invention. The presently disclosed embodiments are therefore to be considered in all respects as illustrative and not restrictive, the scope of the invention being indicated by the appended claims, and not limited to the foregoing description. It will be appreciated that in the claims, the term “comprises/comprising” does not exclude the presence of other elements or steps. In addition, singular references do not exclude a plurality. The terms “a”, “an”, “first”, “second”, etc., do not preclude a plurality.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any amount or range therein.

All percentages, parts and ratios are based upon the total weight of the composition of the present invention, unless otherwise specified. All such weights as they pertain to the listed ingredients are based on the level of the particular ingredient described and, therefore, do not include carriers or by-products that may be included in commercially available materials, unless otherwise specified.

What is claimed is:

1. A method for oral delivery to an infant of a composition containing Bifidobacterium infantis wherein the composition is applied directly to the skin of a caregiver.

2. A method of claim 1 wherein the composition further comprises a prebiotic.

3. A method of claim 2 wherein the prebiotic is an oligosaccharide.

4. A method of claim 2 wherein the prebiotic is a natural oligosaccharide.

5. A method of claim 2 wherein the prebiotic is a synthetic molecule functionally equivalent to a natural oligosaccharide.

6. A method of claim 5 wherein the prebiotic is lacto-N-tetraose (LNT) or 2′-fucosyllactose (2′-FL).

7. A method of claim 1 wherein the composition is lactose free.

8. A method of claim 1 wherein the composition further comprises an antioxidant.

9. A method of claim 1 wherein the composition further comprises a vitamin, mineral or supplement.

10. A method of claim 1 wherein the composition further comprises a food allergen.

11. A method of claim 1 wherein the composition is contained in a soft gel.

12. A method of claim 1 wherein the composition is a semisolid.

13. A method of claim 1 wherein the composition is stable for at least 3 months when stored at 25° C. and 65% relative humidity.

14. A method of claim 1 wherein the composition is stable for at least 6 months when stored at 25° C. and 65% relative humidity.

15. A method of claim 1 wherein the composition is stable for at least 12 months when stored at 25° C. and 65% relative humidity.

16. A method of claim 1 wherein the composition is stable for at least 24 months when stored at 25° C. and 65% relative humidity.

17. A method of claim 1 wherein the composition is anhydrous.

18. A method of claim 1 wherein the composition further comprises an ingredient suitable for skin in need of improving skin barrier function and/or moisturization.

19. A method of claim 1 wherein the composition further comprises an ingredient suitable to soothe irritated skin or protect against skin irritation.

20. A method for oral delivery to an infant of a composition containing Bifidobacterium infantis wherein the composition is applied via a caregiver's finger to one of the gums, teeth or cheek of the infant.

21. A method for oral delivery to an infant of a composition containing Bifidobacterium infantis wherein the composition is applied to or pre-loaded into an artificial nipple or pacifier.

22. A composition for oral delivery to an infant of a composition containing Bifidobacterium infantis wherein the composition is a dissolvable or disintegrable tablet.

23. A composition of claim 22 further comprising one or more of a prebiotic, a food allergen and a vitamin, mineral or supplement.

24. A composition of claim 22 wherein the composition is stable for at least 12 months when stored at 25° C. and 65% relative humidity.

25. A composition of claim 22 wherein the composition is stable for at least 24 months when stored at 25° C. and 65% relative humidity.
